(19) United States
(12) Patent Application Publication
(10) Pub. No.: US 2026/0098847 A1
(43) Pub. Date: Apr. 9, 2026

(54) TEXTURED FILM FOR USE IN A DISSOLUTION ADAPTER AND USES THEREOF

(71) Applicant: University of Connecticut, Farmington, CT (US)

(72) Inventors: Diane J. BURGESS, Mansfield, CT (US); Xiaoyi WANG, Farmington, CT (US)

(21) Appl. No.: 19/350,768

(22) Filed: Oct. 6, 2025

Related U.S. Application Data
(60) Provisional application No. 63/703,225, filed on Oct. 4, 2024.

(51) Int. Cl. G01N 33/15

(52) U.S. Cl. CPC G01N 33/15

(57) ABSTRACT

Disclosed herein is a film for receiving an implant formulation for in vitro testing, the film comprising a roughened surface and a scribed area on the roughened surface for receiving an in situ forming implant formulation; the in situ formulation comprising a pharmaceutically acceptable therapeutic molecule, a biodegradable polymer, a water-miscible solvent, or any combination thereof; and where a surface roughness of the roughened surface is operative to control a surface to volume ratio of the in situ forming implant formulation when tested in a dissolution adapter and/or a cassette such that a Level A in vitro-in vivo correlation is obtained.

### CROSS REFERENCE TO RELATED APPLICATIONS

This application claims priority to U.S. Provisional Application having Ser. No. 63/703,225 filed on Oct. 4, 2024, the entire contents of which are hereby incorporated by reference.

### STATEMENT REGARDING FEDERALLY SPONSORED RESEARCH

This invention was made with government support under 75F40120C00021 awarded by the Food and Drug Administration. The government has certain rights in the invention.

### BACKGROUND OF THE INVENTION

### Field of the Invention

The present disclosure provides textured film for use in adapters for dissolution. More specifically, the present disclosure is directed to a disposable slide/film platform that may be used in a dissolution adapter and a method of employing the same in a United States Pharmacopoeia (USP) dissolution apparatus or a non-Pharmacopoeia dissolution apparatus for conducting in-vitro dissolution, release, and/or testing of formulations.

### Description of Related Art

In vitro-in vivo correlation (IVIVC) is widely recognized as an important tool in the pharmaceutical industry, academia, and among regulatory agencies, as it enables prediction of drug product in vivo performance based on in vitro release testing. A well-established IVIVC can serve as a surrogate for bioequivalence studies, thereby reducing the need for extensive and costly clinical trials. For conventional dosage forms such as oral solid dosage units, successful Level A IVIVCs have been demonstrated and are often used in regulatory submissions.

Despite these advantages, the development of IVIVCs for in situ forming implants has remained elusive. In situ forming implants are injectable liquid formulations that undergo phase separation following administration, forming a solid or semi-solid depot at the injection site. The unique complexity of the drug release mechanisms associated with such systems, including solvent exchange, polymer precipitation, depot morphology evolution, and environmental interaction, results in significant discrepancies between in-vitro and in-vivo drug release profiles. Consequently, IVIVCs for in situ forming implants have not been successfully established to date.

Poly(lactide-co-glycolide) (PLGA) and N-methyl-2-pyrrolidone (NMP)-based in situ forming implants are injectable liquid dosage forms that undergo solidification through phase separation following administration. Phase separation occurs as the NMP solvent diffuses into the surrounding aqueous environment while water simultaneously penetrates into the formulation. This solvent exchange results in precipitation of the PLGA polymer together with the drug, forming a solid depot capable of sustained release over extended periods. The drug release mechanisms associated with in situ forming implants are complex and typically involve: (1) burst release arising from the initial phase separation; (2) diffusion-controlled release dependent on drug and polymer properties as well as the microstructure of the polymer matrix; and (3) degradation-controlled release dominated by PLGA degradation. These mechanisms yield distinct release phases with varying kinetics, allowing in situ forming implants to deliver drugs for weeks to months depending on polymer characteristics.

Since 1998, multiple in situ forming implant products have been approved by the United States Food and Drug Administration (FDA). However, no specific FDA recommendations or standardized compendial dissolution methods exist for these dosage forms. The absence of standardized in vitro release methods, combined with the lack of robust PLGA characterization standards, has hindered the development of both innovator and generic in situ forming implant products. Prior investigations into risperidone in situ forming implants demonstrated that subtle changes in PLGA molecular weight, lactic acid: glycolic acid (L/G) ratio, and polymer end-cap chemistry influence drug release in vitro and in vivo. Higher PLGA molecular weight, higher L/G ratios, and ester end-caps were all associated with extended-release durations. Despite these findings, reproducible in vitro-in vivo correlations (IVIVCs) have not been established due to significant differences between in vitro and in vivo release behavior.

Existing methods such as the direct injection technique suffer from poor reproducibility because the viscous implant solutions form depots with uncontrolled shapes when injected into the release medium. The PVA-Teflon adapter method, developed to improve consistency, permits controlled implant shaping and reproducible data generation. However, the PVA membrane dissolves rapidly in aqueous media, eliminating mechanical pressure and allowing continuous swelling of the depot, which does not mimic the in vivo environment. Furthermore, the adapter yields depots with surface-to-volume ratios significantly lower than those formed in vivo, particularly for risperidone implants that exhibit substantial swelling in the subcutaneous space. This discrepancy in surface-to-volume ratio and water uptake contributes to differences in phase separation kinetics and drug release profiles. In vivo, as much as 90 weight percent (wt %) of NMP may be released within 24 hours, whereas in vitro release using the PVA-Teflon adapter produces an amount of less than 15 wt % NMP release over the same time frame.

Factors such as surface-to-volume ratio, water uptake, mechanical pressure, and phase separation rate are believed to contribute to these discrepancies, but their precise roles have not been conclusively established. Platforms capable of systematically varying these parameters are needed to design dissolution methods that generate biorelevant release profiles. Hydrogel-based systems have been explored, but they require precise preparation and quality control, limiting reproducibility and efficiency. Thus, simple and robust platforms are needed to more closely replicate the in vivo implant environment and support IVIVC development.

Accordingly, there remains a need for improved in vitro release testing methodologies for in situ forming implants that yield more biorelevant drug release data. Such methodologies would facilitate the establishment of IVIVCs, thereby reducing the reliance on extensive in vivo studies and supporting regulatory approval of long-acting injectable products.

### SUMMARY OF THE INVENTION

Disclosed herein is a film for receiving an implant formulation for in vitro testing, the film comprising a roughened surface; a scribed area on the roughened surface for receiving an in situ forming implant formulation; the in situ formulation comprising a pharmaceutically acceptable therapeutic molecule, a biodegradable polymer, a water-miscible solvent, or any combination thereof; and where a surface roughness of the roughened surface is operative to control a surface to volume ratio of the in situ forming implant formulation when tested in a dissolution adapter and/or a cassette such that a Level A in vitro-in vivo correlation is obtained.

Disclosed herein is a method of establishing an in vitro-in vivo correlation (IVIVC) for a pharmaceutically acceptable therapeutic molecule, the method comprising disposing an in situ formulation comprising the pharmaceutically acceptable therapeutic molecule, a biodegradable polymer and a solvent into a roughened surface of a film, the roughened surface comprising a scribed receptacle that is operative to confine the formulation to a defined geometry; disposing the film into a dissolution adapter where it is subjected to a dissolution solvent; subjecting the dissolution adapter to in vitro release testing in a dissolution vessel selected from USP Apparatus 1, USP apparatus 2, USP Apparatus 4, or a screw-capped bottle in a water bath shaker, and collecting medium samples over time to determine the in vitro release profile; administering the formulation to a subject and collecting plasma samples over time to determine an in vivo pharmacokinetic profile; processing the in vivo pharmacokinetic profile by deconvolution to determine an in vivo absorption profile; and correlating the in vitro release profile with the in vivo absorption profile to establish a Level A IVIVC.

### BRIEF DESCRIPTION OF THE DRAWINGS

The features and advantages of the invention are apparent from the following description taken in conjunction with the accompanying drawings in which:

FIGS. 1A and 1B are schematic depictions of films that have a receptacle with a roughened surface; the open receptacle is operative to receive a liquid formulation testing and evaluation;

FIG. 2 depicts a sandblasted glass slide adapter with laser created ring and cassette;

FIGS. 3A and 3B show the impact of sample loading dose on in vitro risperidone (A) and NMP (B) release profiles of formulation F #0 tested using the glass slide adapter method; F #0 was prepared using PLGA (MW: 24.2 kDa; L/G ratio: 80/20; blockiness: 2.15; acid end-cap). Testing conditions: USP 2, 0.01 M PBS, pH 7.4, 50 rpm, 37° C. n=3;

FIG. 4 shows degradation profiles of formulation F #0 using different adapter-based dissolution methods. F #0 was prepared using PLGA (MW: 24.2 kDa; L/G ratio: 80/20; blockiness: 2.15; acid end-cap); n=3.

FIGS. 5A, 5B and 5C show comparison of in vitro release profiles of different risperidone implant formulations obtained using a glass slide adapter-USP2 method (0.01 M PBS, pH 7.4, 50 rpm, 37° C.) and their deconvoluted in vivo absorption profiles. (A) F #2 was prepared using PLGA (MW: 17.8 kDa; L/G ratio: 80/20; blockiness: 2.50; acid end-cap); (B) F #8 was prepared using PLGA (MW: 31.1 kDa; L/G ratio: 80/20; blockiness: 2.10; acid end-cap); (C) Perseris PLGA has a MW of 22.3 kDa, L/G ratio of 80/20, blockiness of 2.53 and acid end-cap;

FIG. 6 shows in vitro release profiles of NMP from different risperidone implant formulations obtained using a glass slide adapter-USP2 method (⅓ loading dose). Testing conditions: 0.01 M PBS, pH 7.4, 50 rpm, 37° C.;

FIG. 7 shows a level A IVIVC model of risperidone implants created using release data obtained from the glass slide adapter based USP 2 method. Fit dissolution model: Makoid-Banakar model; correlation: Fabs-Diss (Tvivo); and

FIGS. 8A through 8H shows internal and external validation of the IVIVC model of risperidone implants established using release data obtained from glass slide adapter based USP 2 method. (A-D) Experimental and predicted in vivo release profiles of risperidone implant formulations. (E-H) Experimental and predicted PK profiles. Fit dissolution model: Makoid-Banakar model; correlation: Fabs=Diss (Tvivo); internal validation: Formulations F #0, F #2, F #8; external validation: Perseris.

### DETAILED DESCRIPTION OF THE INVENTION

Disclosed herein is an in vitro release testing platform (hereinafter “platform”) for evaluating drug release from an in situ forming implant, the platform comprises a dissolution adapter that contains a film with a roughened surface. The film has an open receptacle disposed thereon that is used to delineate an area of the film on which a liquid formulation is to be placed for testing and evaluation. The receptacle is configured to receive the liquid formulation comprising a pharmaceutically acceptable therapeutic molecule, a biodegradable polymer and a water-miscible solvent, or any combination thereof (hereinafter the formulation), wherein the receptacle constrains the geometry of the liquid formulation formed upon contact with the roughened surface. Without being limited to theory, surface roughness affects the receding and the advancing contact angles, with a larger effect on the receding contact angle (decreasing). Surface roughness effectively increases the surface area and the liquid gets trapped in the crevices created on the surface. This has the effect of holding or pinning the liquid to the surface.

The term pharmaceutically acceptable therapeutic molecule includes salts, isomers and derivatives thereof. The receptacle is of a size effective (i.e., a surface area or a volume) to receive the liquid formulation so that an in vitro-in-vivo correlation is established. The film may comprise a polymer, a ceramic, a metal or a combination thereof. The geometry of the liquid formulation on the receptacle provides a surface-to-volume ratio and water uptake profile that closely mimics in vivo conditions.

Disclosed herein too is a method of manufacturing a film for use in a dissolution adapter that comprises roughening a surface of a film and delineating a receptacle on the roughened surface. The receptacle is an open receptacle that is operative to define a deposition area for deposition of the formulation. The roughness has an average surface roughness (Ra) of about 0.1 to about 5 micrometers. The method comprises subjecting the surface to controlled abrasive blasting using fine grit alumina, glass beads, or similar abrasive media; alternatively, the method may include mechanical abrasion using silicon carbide abrasive papers, polishing films, or lapping slurries of suitable grit size. In other embodiments, the method may include chemically etching the surface of the film using acidic or alkaline solutions under controlled conditions, or electrochemical etching at low current densities to produce micro-roughened features within the specified Ra range. In other embodiments, the method may include pore-forming technologies such as phase inversion, tack etching, electrospinning, sintering, stretching, foaming & gas expansion to produce surface roughness. In further embodiments, the method may include physical texturing by laser ablation, plasma etching, or ion beam sputtering under selected processing parameters to provide the desired surface topology.

Disclosed herein are: 1) identification of useful attributes of in vitro release testing methods that may contribute to differences in in vitro and in vivo drug release from in situ forming implants; and 2) refinement of the in vitro release method, with the aim of developing Level A IVIVCs for risperidone implants.

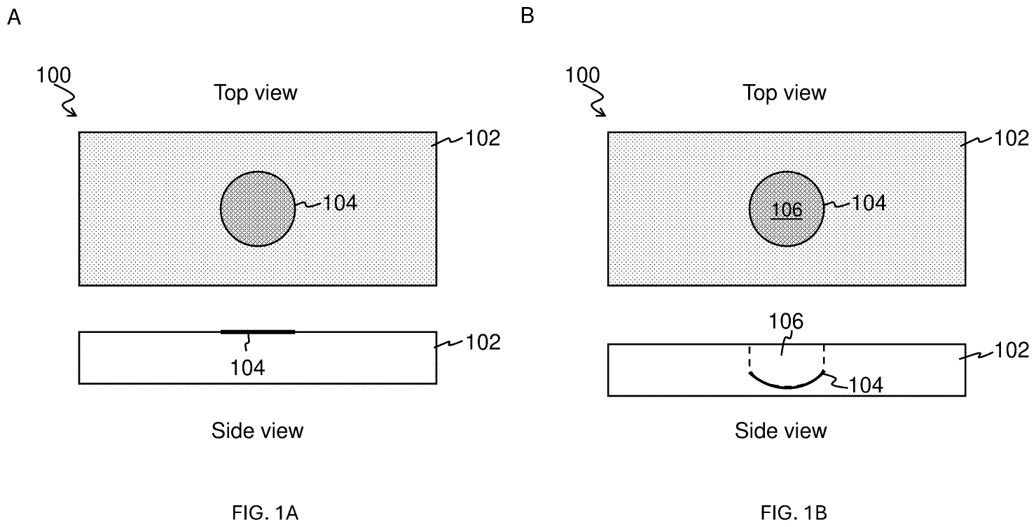

FIG. 1A is an exemplary depiction of the film 100 (that is placed in a dissolution adapter) for evaluating drug release from an in situ forming implant formulation (also referred to herein as the “formulation”). The film 100 comprises a surface 102 that is roughened by any of the foregoing methods to have an average surface roughness (Ra) of about 0.1 to 5 micrometers, preferably about 0.5 to 3 micrometers, and more preferably about 1 to 2 micrometers. The roughened surface promotes adhesion of the formulation and prevents migration during testing, while optional laser-etched reference markings 104 (referred to herein as the open receptacle 104) may be used to define deposition areas and ensure reproducibility. In an embodiment, the film 100 comprises an open receptacle 104 with a roughened surface having an average surface roughness of about 0.1 to 5 micrometers. In other words, the entire surface 102 of film 100 may be roughened or alternatively, only a surface of the receptacle 104 may be roughened, while the remainder of the surface 102 is smooth. In an embodiment, the area outside the receptable 104 may be smooth, while the area inside the receptacle 104 is roughened.

FIG. 1B is another exemplary depiction of the film 100 that has a surface 102 that may or may not be roughened. The receptacle 104 is located at the bottom of a cavity 106. The cavity 106 protrudes into the film 100 from surface 102. The floor of the cavity 106 is roughened and functions as the open receptacle 104.

The receptacle 104 has a diameter of about 0.5 to 10 centimeters, preferably about 1.5 to 8 centimeters. In an embodiment, the open receptacle 104 is a created ring with a plane surface, a cylindrical well, or a round bottom concave surface. The open receptacle 104 on the film 102 does not have to be created by the laser, but other suitable methods may be used as well. The receptacle 104 does not have to be circular in cross-sectional area; it may have a square, rectangular, ellipsoidal, triangular, polygonal (where the number of sides “n” is greater than 4) cross-sectional area. In an embodiment, the cross-sectional area may be irregular. In certain embodiments, the cross-sectional area of the receptacle may be irregular and defined by the enclosed boundary of the surface profile, which may be determined by mathematical integration, geometric decomposition, or numerical approximation of the region lying within the perimeter.

In certain embodiments, the receptacle 104 (also referred to as a reference ring or deposition circle) formed on the surface 102 of the film 100 may be created by methods other than laser etching. For example, the ring may be formed by mechanical engraving, machining, or scribing, wherein a tool cuts or inscribes a shallow groove in the surface. In another embodiment, the receptacle may be formed by chemical etching, such as contacting a masked portion of the surface with an acid or alkaline etchant to selectively roughen or recess the ring region. In further embodiments, the receptacle may be produced by abrasive blasting, including sandblasting or bead blasting through a stencil to create a circular pattern of increased opacity or roughness. Alternatively, the receptacle may be defined by deposition or printing, including screen printing, inkjet printing, or sputter deposition of a durable ink, resin, or thin film material through a mask to produce a contrasting visible feature. In yet other embodiments, the receptacle may be embossed or molded into the surface by thermal pressing, ultrasonic embossing, or other deformation processes, particularly where the film 100 comprises a polymeric substrate. Each of these approaches provides a durable and defined reference feature on the surface of the film 100 suitable for controlling the geometry and placement of the formulation.

In certain embodiments, the dissolution adapter comprises a slide or film having a surface roughened region with a scribed or etched area operative to confine deposition of an in situ forming implant formulation. The defined scribed area establishes a reproducible surface-to-volume ratio of the deposited liquid formulation upon solvent exchange and solidification, thereby producing a depot geometry that mimics the depot formed in vivo following subcutaneous or intramuscular injection. By controlling the spatial extent of the liquid formulation during in vitro testing, the scribed area ensures that solvent diffusion, polymer precipitation, and drug release kinetics occur in a manner that is predictive of in vivo performance, thereby facilitating the development of Level A in vitro in vivo correlation.

The surface roughness of the scribed or blasted region further enhances adhesion of the liquid formulation by creating micro-scale interlocking features that lower the contact angle and resist displacement under hydrodynamic conditions. The degree of roughness may be tailored to the viscosity and surface tension of the formulation; for example, lower viscosity, solvent-rich formulations may require a more aggressively roughened or deeply scribed substrate to prevent detachment, whereas higher viscosity formulations may adhere effectively with less surface modification. In this manner, the roughness is functionally matched to the rheological properties of the formulation to ensure anchoring and reproducibility. The roughening may create a texture that is random or non-random. Non-random features include periodic features that have well-defined geometries (features that are defined by Euclidean geometry) that are repeated across the surface of the film (or the receptacle).

Optionally, the combination of defined scribed area size and tailored surface roughness yields depots that undergo solvent exchange and phase inversion at rates closely resembling those observed in vivo, producing release profiles that exhibit comparable burst release, diffusion-controlled release, and erosion-controlled phases. Accordingly, the described surface treatment of the adapter provides a reproducible and scientifically grounded method for aligning in vitro release behavior with in vivo pharmacokinetics, thereby improving predictability and enabling regulatory-acceptable IVIVC models.

The film 100 may comprise a polymer, a ceramic, a metal, or a combination thereof. The film may be rigid or flexible. When the film is flexible, it may be disposed on a substrate (not shown) for support. The material of the film is preferably resistant to chemical attack or dissolution by the formulation. The film can have a thickness of about 1 micrometer to about 5000 micrometers, preferably about 100 to 3000 micrometers, and more preferably about 200 to 1000 micrometers. The receptable 104 may generally include an area on the surface of the film that is roughened, or alternatively, may be a surface at the bottom of a cavity that protrudes into the film from the surface 102.

Organic polymers used in the film 100 may be selected from a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, a polyelectrolyte (polymers that have some repeat groups that contain electrolytes), a polyampholyte (a polyelectrolyte having both cationic and anionic repeat groups), an ionomer, or the like, or a combination comprising at last one of the foregoing organic polymers. The organic polymers have number average molecular weights greater than 10,000 grams per mole, preferably greater than 20,000 g/mole and more preferably greater than 50,000 g/mole.

Examples of suitable polymers for use in the film 102 include a polyacrylic, a polycarbonate, a polyalkyd, a polystyrene, a polyolefin, a polyester, a polyamide, a polyaramid, a polyamideimide, a polyarylate, a polyurethane, an epoxy, a phenolic, a polysiloxane, a polyarylsulfone, a polyethersulfone, a polyphenylene sulfide, a polysulfone, a polyimide, a polyetherimide, a polytetrafluoroethylene, a polyetherketone, a polyether ether ketone, a polyether ketone ketone, a polybenzoxazole, a polyoxadiazole, a polybenzothiazole, a polybenzothiazinophenothiazine, a polypyrazinoquinoxaline, a polypyromellitimide, a polyguinoxaline, a polybenzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polycarborane, a polyoxabicyclononane, a polydibenzofuran, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polythioester, a polysulfone, a polysulfonamide, a polyurea, a polyphosphazene, a polysilazane, a polyolefin, or the like, or a combination thereof. Preferred polymers are polyolefins, polytetrafluoroethylene, polysiloxanes, polyesters, or a combination thereof.

Ceramic films include metal oxides, metal carbides, metal nitrides, metal borides, metal silicides, metal oxycarbides, metal oxynitrides, metal boronitrides, metal carbonitrides, metal borocarbides, or the like, or a combination thereof. Examples of ceramics that may be used as the film include silicon dioxide (e.g., glass slides), aluminum oxide, titanium dioxide, zirconium dioxide, indium tin oxide, antimony tin oxide, cerium oxide, cadmium-oxide, titanium nitride, silicon nitride, aluminum nitride, titanium carbide, silicon carbide, titanium niobium carbide, stoichiometric silicon boride compounds (SiBₙ, where n=14, 15, 40, and so on) (e.g., silicon triboride, SiB₃, silicon tetraboride, SiB₄, silicon hexaboride, SiB₆, or the like), or the like, or a combination thereof. A preferred film is a silicon dioxide slide (e.g., a glass slide).

Metal films include iron, copper, titanium, aluminum, vanadium, molybdenum, nickel, cobalt, silicon, gallium, indium, thallium, or the like, or a combination thereof. Suitable alloys are stainless steel, carbon steel, titanium-aluminum alloys, ferroalloys, ferroboron, ferrochrome (chromium), ferromagnesium, ferromanganese, ferromolybdenum, ferronickel, ferrophosphorus, ferrotitanium, ferrovanadium, ferrosilicon, Al—Li (aluminum, lithium, sometimes mercury), Alnico (aluminum, nickel, copper), Duralumin (copper, aluminum), Magnalium (aluminum, 5% magnesium), Magnox (magnesium oxide, aluminum), Nambe (aluminum plus seven other unspecified metals), Silumin (aluminum, silicon), Billon (copper, silver), Brass (copper, zinc), Calamine brass (copper, zinc), Chinese silver (copper, zinc), Dutch metal (copper, zinc). Gilding metal (copper, zinc), Muntz metal (copper, zinc), Pinchbeck (copper, zinc), Prince's metal (copper, zinc), Tombac (copper, zinc), Bronze (copper, tin, aluminum, or any other element), Alumel (nickel, manganese, aluminum, silicon), Chromel (nickel, chromium), Cupronickel (nickel, bronze, copper), German silver (nickel, copper, zinc), Hastelloy (nickel, molybdenum, chromium, sometimes tungsten), Inconel (nickel, chromium, iron), Monel metal (copper, nickel, iron, manganese), Mu-metal (nickel, iron), Ni—C (nickel, carbon), Nichrome (chromium, iron, nickel), Nicrosil (nickel, chromium, silicon, magnesium), Nisil (nickel, silicon), Nitinol (nickel, titanium, shape memory alloy), or the like, or a combination thereof.

The surface may be roughened by various methods such as ion beam etching, roughening a surface to achieve an average surface roughness (Ra) of about 0.1 to 5 micrometers comprises subjecting the surface to controlled abrasive blasting using sand, fine grit alumina, glass beads, or a combination thereof, the method may include mechanical abrasion using silicon carbide abrasive papers, polishing films, or lapping slurries of suitable grit size. In other embodiments, the method includes chemically etching the surface using acidic or alkaline solutions under controlled conditions, or electrochemical etching at low current densities to produce micro-roughened features within the specified Ra range. In other embodiments, the method may include pore-forming technologies such as phase inversion, track etching, electrospinning, sintering, stretching, foaming & gas expansion to produce surface roughness. In further embodiments, the method comprises physical texturing by laser ablation, plasma etching, or ion beam sputtering under selected processing parameters to provide the desired surface morphology.

In an embodiment, the surface of the film 102 is roughened by techniques that include sand blasting or bead blasting. Sand blasting or bead blasting is a surface treatment process in which a stream of abrasive particles is propelled at high velocity toward a substrate to alter its surface characteristics. The abrasive medium may include sand, alumina, glass beads, or other particulates, and is delivered under compressed air or liquid pressure. Upon impact, the abrasive erodes, peens, or roughens the surface, thereby increasing its average surface roughness and producing a matte or satin finish. The resulting surface modification may improve adhesion of coatings, enhance wettability, increase mechanical interlocking, or provide defined topographies suitable for subsequent functionalization. In certain embodiments, bead blasting with glass microspheres may be employed to produce smoother, less angular textures, while sand blasting with angular particles may yield higher roughness and sharper profiles. Control of blasting parameters such as particle size, pressure, angle of incidence, and exposure duration permits tailoring of the surface finish to achieve desired performance characteristics.

In particular, a sandblasted glass slide platform (also sometimes referred to as an adapter) is developed to receive a liquid formulation, maintain the formulation in a defined geometry, and thereby control the surface-to-volume ratio of the solidified formulation in a manner that more closely mimics in vivo conditions. The sandblasted surface promotes adhesion of the formulation and prevents migration during testing, while optional laser-etched reference markings may be used to define deposition areas and ensure reproducibility. Using this platform, an in vitro dissolution method is established that successfully generated a Level A in vitro in vivo correlation (IVIVC) for risperidone implants, permitting prediction of in vivo performance from in vitro release data.

In an embodiment, the film 100 (of FIG. 1A or 1B) may be used in a dissolution adapter (not shown). A dissolution adapter is a specialized accessory used in pharmaceutical testing to hold a non-tablet dosage form (e.g., a film, gel, injectable depot, or implant) in place during in vitro dissolution testing. In certain embodiments, the present disclosure provides for a dissolution adapter that is operative to secure the film 100 within a controlled dissolution environment. The dissolution adapter may be configured for use in a United States Pharmacopocia (USP) Apparatus 2 vessel, such as a paddle dissolution system, wherein the film with the formulation disposed thereon is positioned at the bottom of the vessel to maintain the dosage form in a fixed geometry relative to the stirring paddle and dissolution medium. This ensures the formulation stays in the hydrodynamic field without floating away. As the dissolution medium circulates, the formulation releases drug molecules into solution. The fixed geometry ensures that dissolution follows a reproducible pattern, allowing meaningful comparisons of release profiles.

In other embodiments, the dissolution adapter may be configured for placement in a screw-capped bottle disposed within a water bath shaker, thereby permitting agitation under controlled thermal and hydrodynamic conditions, while preventing uncontrolled migration of the formulation within the bottle. In yet further embodiments, the adapter may be adapted for use in a USP Apparatus 4 flow-through cell, wherein the dissolution medium is continuously perfused through the cell to simulate dynamic physiological flow, and the dissolution adapter maintains the dosage form in a reproducible position within the fluid path.

The dissolution adapter thereby provides flexibility across multiple compendial testing platforms, enabling reproducible assessment of in-vitro release characteristics under both static and dynamic conditions. By ensuring adhesion and geometric control of the formulation, the adapter minimizes variability associated with uncontrolled movement, floating, or disintegration of the sample within the dissolution medium. As a result, the adapter facilitates improved correlation between in vitro release data and in vivo performance, while also providing a standardized platform suitable for regulatory testing, quality control, and method development across different USP dissolution apparatuses.

In some embodiments, the present disclosure relates to a Level A in vitro in vivo correlation (IVIVC). A Level A IVIVC is defined as a point-to-point relationship between the in-vitro dissolution profile of a pharmaceutical dosage form and the in vivo absorption profile of the same dosage form, wherein substantially the entire plasma concentration-time curve of the drug can be predicted from the in vitro dissolution data. Such a correlation provides the highest level of predictability and regulatory acceptance, as it establishes that the rate and extent of drug dissolution in a controlled laboratory test directly correspond to the rate and extent of systemic drug absorption in a human or animal subject. In particular embodiments, the Level A IVIVC permits the use of in vitro dissolution testing as a surrogate for certain in vivo bioavailability or bioequivalence studies, thereby enabling the reduction of clinical testing requirements, facilitating regulatory approval, and supporting post-approval changes such as scale-up, site transfer, or formulation modification without the necessity of repeating extensive in vivo trials.

In one embodiment, in one method of evaluating a drug release from an in situ forming implant, the method comprises providing a dissolution testing platform comprising a non-dissolvable shape-controlling film having a receptacle of defined geometry. The surface of the receptacle is roughened. A liquid formulation comprising a pharmaceutically acceptable therapeutic molecule, a biodegradable polymer and a water-miscible solvent is introduced into receptacle. The film containing the formulation is immersed in a dissolution adapter that contains an aqueous release medium under dissolution testing conditions. Drug release is measured from the formulation over time, wherein the adapter constrains the geometry of the formulation to provide a surface-to-volume ratio and water uptake profile that more closely mimics in-vivo conditions as compared to a direct injection method.

In certain embodiments, a method of establishing a Level A in vitro in vivo correlation (IVIVC) comprises disposing an in situ forming implant formulation onto a slide or film positioned within a dissolution adapter, the slide or film comprising a roughened or scribed surface operative to confine and anchor the formulation. The adapter is then introduced into a dissolution vessel selected from a USP Apparatus 1, USP apparatus 2, USP Apparatus 4, or a screw-capped bottle in a water bath shaker. The formulation is subjected to release testing under controlled hydrodynamic conditions, and aliquots of the dissolution medium are withdrawn at predetermined time points and analyzed by high-performance liquid chromatography (HPLC), ultraviolet spectrophotometry, or liquid chromatography-mass spectrometry (LC-MS) to generate an in vitro cumulative drug release profile.

The method further comprises administering the same formulation to an animal model or human subject to obtain in vivo pharmacokinetic data, including plasma concentration-time profiles, Cₘₐₓ, Tₘₐₓ, and area under the curve (AUC). The pharmacokinetic data are mathematically processed by deconvolution methods, including Wagner-Nelson or numerical deconvolution, to determine an in vivo absorption profile expressed as percent absorbed versus time. The in vitro release profile is then correlated with the in vivo absorption profile in a point-to-point manner to generate a Level A IVIVC.

In certain embodiments, the method further comprises evaluating the predictability of the IVIVC model by calculating the percentage prediction error (% PE) for pharmacokinetic parameters, including Cₘₐₓ and AUC, for formulations used in model development (internal predictability) and for formulations not used in model development (external predictability). The IVIVC is deemed acceptable when the mean absolute % PE is less than about 10% and the individual % PE for each formulation is less than about 15%, thereby demonstrating that the dissolution adapter and testing protocol are operative to produce reliable Level A IVIVCs suitable for formulation development and regulatory submission.

In certain embodiments, the sample tested within the dissolution adapter comprises a pharmaceutically acceptable therapeutic molecule selected from the genus of small molecules, peptides, or proteins. Suitable species include antipsychotics such as risperidone; opioid antagonists such as naltrexone; gonadotropin-releasing hormone (GnRH) agonists such as leuprolide acetate, goserelin, and triptorelin; peptide therapeutics such as exenatide and octreotide; partial opioid agonists such as buprenorphine; anti-inflammatory and analgesics such as dexamethasone, diclofenac, and ketoprofen; antibiotics such as ciprofloxacin, gentamicin, and amoxicillin; and biologics including insulin, interferons, and bovine serum albumin (BSA) as a model protein. These therapeutic molecules, when incorporated into in situ forming implants or depot formulations, may be tested in vitro within the dissolution adapter to generate drug release profiles that are subsequently correlated with in vivo pharmacokinetic data to establish Level A IVIVCs.

In certain embodiments, the biodegradable polymer comprises a member selected from the genus of synthetic aliphatic polyesters, polyanhydrides, poly(ortho esters), or naturally derived biopolymers, and species thereof. Suitable species of aliphatic polyesters include poly(lactic acid) (PLA), poly(glycolic acid) (PGA), poly(lactide-co-glycolide) (PLGA), polycaprolactone (PCL), and poly(trimethylene carbonate) (PTMC). Suitable species of polyanhydrides include poly(sebacic anhydride), poly(fatty acid dimer anhydride), and poly(anhydride-co-imide). Suitable species of poly(ortho esters) include POE-I, POE-II, POE-III, and POE-IV families having backbones with biodegradable ester linkages. Suitable species of polycarbonates include aliphatic polycarbonates such as poly(trimethylene carbonate), as well as tyrosine-derived polycarbonates such as poly(desaminotyrosyl-tyrosine ethyl ester carbonate). In certain embodiments, the biodegradable polymer may also be derived from a natural source, including chitosan, alginate, gelatin, collagen, hyaluronic acid, dextran, or pullulan. Other useful species include polydioxanone (PDO), poly(butylene succinate) (PBS), and polyhydroxyalkanoates (PHAs), such as poly(3-hydroxybutyrate) (PHB) and copolymers thereof. These polymers, whether employed individually or in combination, are operative to undergo controlled hydrolytic or enzymatic degradation, thereby permitting the establishment of reproducible in vitro release profiles that can be correlated with in vivo pharmacokinetic data to develop Level A in vitro-in vivo correlations for long-acting drug delivery systems, including but not limited to risperidone implants.

In certain embodiments, the solvent system employed for the formulation of the biodegradable implant or depot comprises a member selected from the genus of polar aprotic solvents, hydrophilic organic solvents, or carbonate esters, and species thereof. Suitable species of formulation solvents include N-methyl-2-pyrrolidone (NMP), dimethyl sulfoxide (DMSO), dimethylacetamide (DMAc), tetrahydrofuran (THF), ethanol, benzyl alcohol, triacetin (glycerol triacetate), and propylene carbonate. In certain embodiments, the dissolution or release medium comprises an aqueous buffer selected from the genus of physiologically relevant buffered saline systems, including species such as phosphate-buffered saline (PBS), simulated body fluid (SBF), Hank's balanced salt solution (HBSS), tris buffer, acetate buffer, or carbonate buffer. The solvent system may further comprise additives selected from the genus of surfactants, co-solvents, or complexing agents, and species thereof, including polysorbate 80 (Tween 80), sodium dodecyl sulfate (SDS), Pluronic F68, ethanol, polyethylene glycol (PEG 400), and hydroxypropyl-β-cyclodextrin. The foregoing solvents and solvent systems are operative to dissolve or disperse the biodegradable polymer and drug during formulation, and to maintain sink conditions and physiologically relevant solubility during in vitro release testing, thereby facilitating the establishment of reproducible release profiles that correlate with in-vivo pharmacokinetics.

The film with the surface roughened receptable is exemplified by the following non-limiting examples.

### EXAMPLES

### Example 1

### Methods and Formulation Preparation

Seven risperidone in situ forming implant formulations were prepared using different poly(lactic-co-glycolic acid) (also called poly(lactide-co-glycolide) (PLGAs). The composition and corresponding PLGA attributes of each formulation are listed in Table 1. In brief, PLGA and N-methyl pyrrolidone (NMP) were precisely weighed into 3 milliliters (mL) glass vials at a PLGA/NMP weight ratio of 228:282 and stirred overnight at room temperature to obtain uniform solutions. Before using, 510 milligram (mg) of PLGA/NMP solutions and 90 mg of risperidone were separately weighed into sterilized HENKE-JECT all plastic syringes to obtain a liquid syringe containing PLGA/NMP solution and a powder syringe containing risperidone. Homogeneous suspensions were obtained through mixing the contents in the two syringes for ˜100 cycles after coupling them together through a Nordson Medical female luer thread coupler. The suspensions were then drawn into the liquid syringe and attached with a needle for in vitro and in vivo injection.

TABLE 1

| Formulation | | | |
|---|---|---|---|
| No. (F#) | PLGA | Solvent | Drug |
| 0 | 228 mg PLGA (MW: 24.2; L/G | 282 mg | 90 mg |
| | ratio: 80/20; Blockiness: 2.15; | NMP | Risperidone |
| | Acid end-cap) | | |
| 2 | 228 mg PLGA (MW: 17.8; L/G | 282 mg | 90 mg |
| | ratio: 80/20; Blockiness: 2.50; | NMP | Risperidone |
| | Acid end-cap) | | |
| 8 | 228 mg PLGA (MW: 31.1; L/G | 282 mg | 90 mg |
| | ratio: 80/20; Blockiness: 2.10; | NMP | Risperidone |
| | Acid end-cap) | | |

Note:
MW = molecular weight; L/G ratio = lactic acid/glycolic acid ratio.

### In-Vitro Drug and NMP Release Testing Using Shape-Controlling Glass Slide Adapter Method

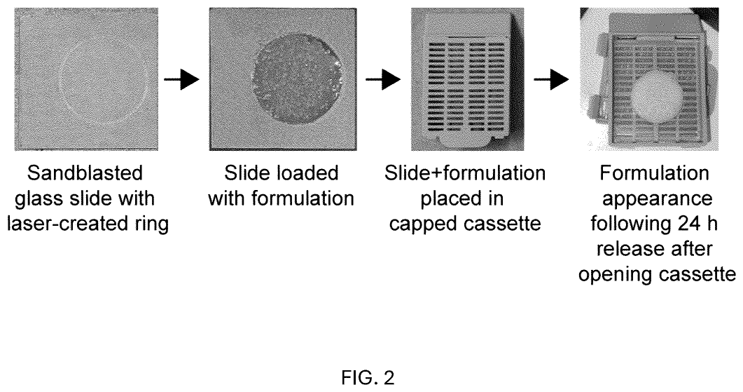

Glass sides with a laser-created ring with a diameter of 1.6 centimeter to help control the shape of the risperidone implant suspension is illustrated in FIG. 2. Different amounts of implant formulations (110 to 120 mg: full dose; 55-60 mg: ½ dose and ˜35 mg: ⅓ dose) were weighed into the ring and spread evenly within the ring. The adapter was then added into cassettes and immersing into USP apparatus 2 vessels for release testing (37° C., 50 rpm). All the release studies were conducted in triplicate.

### Degradation

Degradation behavior of formulations in different adapters with different doses was tested under the conditions described in. “In vitro drug and NMP release testing using shape-controlling glass slide adapter method.” At days 1, 7 and 20 following immersing the adapters along with the formulations into the release medium, the solidified formulation depots were collected, patted to remove the release medium on the surface and weighed in the wet state (W_{Wet}). The dried mass (W Dried) of the formulation depots were obtained by weighing them again following 72 hours freeze-drying. The water uptake (%) compared with the dried depot was calculated using equation (2) below. Experiments were conducted in triplicate.

$$Water Uptake ( \% ) = \frac{{W}_{Wet} - {W}_{D r i e d}}{{W}_{D r i e d}} * 100 \% \qquad ( 2 )$$

The samples collected as well as the in vivo samples from rabbits were weighed into glass vials. 1.5 mL of tetrahydrofuran was added into each vial to dissolve the PLGA polymer through overnight shaking to obtain polymer solutions with a polymer concentration of ˜2 mg/mL. The samples were filtered using a 0.45 μm PTFE filter and applied for MW analysis using a gel permeation chromatography system (GPC). The GPC was equipped with an Agilent 1100 HPLC system with RI detection. Single Agilent Mixed-D PL gel column for SEC was used to separate molecules with different MW. Polystyrene standards ranging from ˜500 Daltons (Da) to ˜500,000 Da (Agilent PS-2 calibration standard kits) were used to build a calibration curve. A 70 kiloDalton (kDa) polystyrene standard was utilized to assess system suitability.

### IVIVC Analysis

An IVIVC for the risperidone implant formulations was developed following the principles outlined in the U.S. FDA IVIVC guidance on extended-release oral dosage forms. The average in-vivo plasma profiles of the risperidone PLGA implants were deconvoluted using Phoenix-8.3 software based on the plasma concentration-time data in a previous publication (X. Wang, Q. B., et al., In situ forming risperidone implants: Effect of PLGA attributes on product performance, Journal of Controlled Release: 361 (2023) 777-791). The Phoenix-8.3 software was also used for the IVIVC analysis for the risperidone implant formulations.

### In Vitro Release Testing Based on Glass Slide Adapter

Three surface-to-volume ratios, 20, 40 and 60 cm⁻¹ were investigated through loading of about 110 to 120 mg (full dose), about 60 mg (½ dose) and about 35 mg (⅓ dose) formulations on the ring of glass sides (with diameter of 1.6 cm). The formulations formed a thin disk-shaped depot after being placed in the release medium for 24 hours (see FIG. 2).

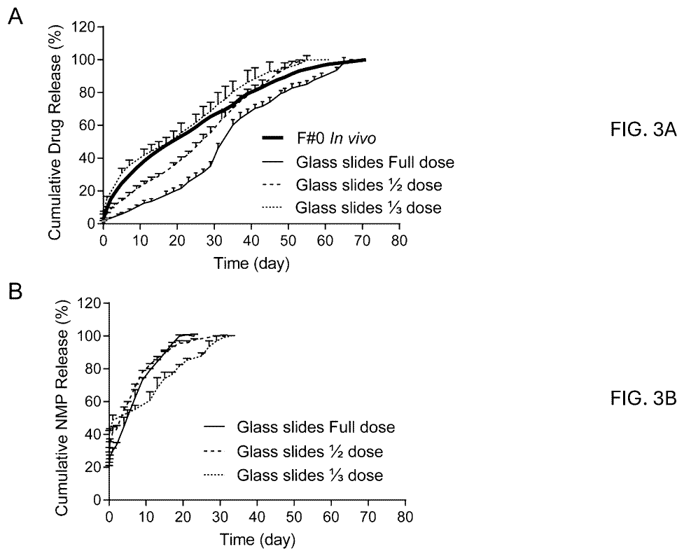

As demonstrated in FIG. 3(B), all three doses tested released ˜40% of NMP within 24 h. In addition, there is a tendency that with the decrease in the surface-to-volume ratio, the NMP release rate within 24 h increases. For the drug release FIG. 3(A), increasing the surface-to-volume ratio through decreasing the sample loading dose appeared to accelerate the diffusion-dominated phase and slow down the degradation-facilitated phase. This may be due to more efficient exchange of materials, and consequently less monomer accumulation in the lower dose depot as a result of the reduced thickness of the depot. Accordingly, the pH would be higher in the inner part of the depot increasing the polymer degradation rate as PLGA is known to undergo acid catalyzed hydrolysis. The increased polymer degradation was confirmed and is discussed in detail below.

A bio-relevant drug release profile was achieved through decreasing the sample loading dose to about 35 mg, which resulted in depots with surface-to-volume ratios of 60 cm⁻¹ (a surface-to-volume ratio close to that of the in vivo depots). The f2 values obtained on comparing the in vitro and in vivo release profiles are 35.64, 49.02 and 64.55 for the full dose, ½ dose and ⅓ dose, respectively, confirming the greater similarity between the in-vitro and in-vivo release profiles with the decrease of the sample loading dose.

### Degradation Profiles of Formulation F #0

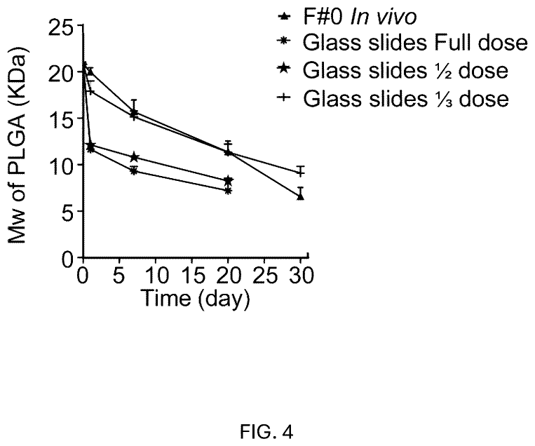

FIG. 4 shows that the degradation profiles of the formulation F #0 were surface-to-volume ratio dependent:the higher the surface-to-volume ratio, the slower the degradation rate. The in-vitro depot formed with the bio-relevant surface-to-volume ratios and generating bio-relevant drug release profiles followed a similar degradation process to that of the in vivo drug depot.

In Vitro Release Testing of Risperidone Implants with Different PLGA Attributes

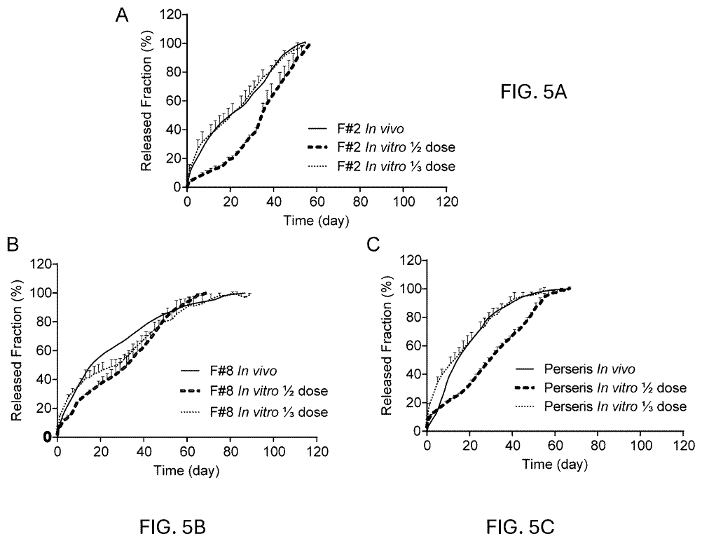

Perseris and risperidone implants prepared using PLGAs with different attributes (molecular weight) were tested using the glass slide adapter method. Two doses, 55 to 60 mg (½ dose) and 35 mg (⅓ dose) were investigated. This study was performed to confirm whether the method optimized based on the representative formulation F #0 was suitable for other risperidone formulations, and whether the impact of dose was the same when tested using other risperidone formulations. Table 2 shows f2 test for in vitro and in vivo release data in FIGS. 5A, 5B and 5C of different formulations at different doses. According to FIGS. 5A, 5B and 5C and Table 2, at ⅓ dose, the glass slide adapter method replicated the in vivo release profiles for all the three formulations tested. F #2 and F #8 have the same PLGA L/G ratio as formulation F #0 but with slightly different MWs (18 KDa, 22 KDa and 29 KDa, for F #2, F #0 and F #8, respectively). Perseris has similar PLGA properties to formulation F #0.

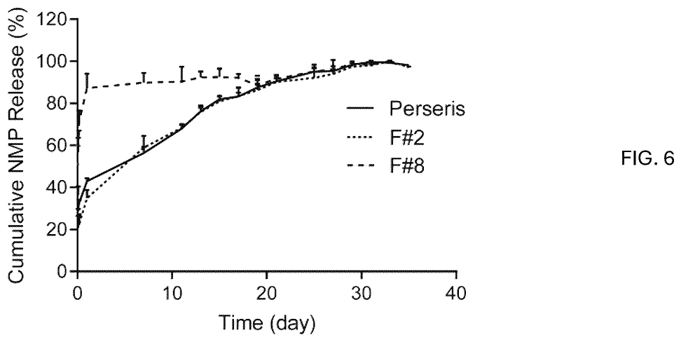

The phase separation rate in FIG. 6 showed some correlation with the hydrophilicity of the PLGA polymers. At ⅓ dose, the formulations prepared using polymers with higher hydrophilicity PLGA (F #2 and Perseris) had slower initial NMP release rates (about 30 to 50%) while those prepared using polymers with lower hydrophilicity PLGA (F #8) demonstrated faster initial NMP release rates within 24 hours (70 to 90%).

TABLE 2

| Formulations | ½ dose | ⅓ dose |
|---|---|---|
| F#2 in vitro vs in vivo | 29.28 | 64.20 |
| F#8 in vitro vs in vivo | 27.67 | 59.53 |
| Perseris in vitro vs in vivo | 33.31 | 58.23 |

### IVIVC

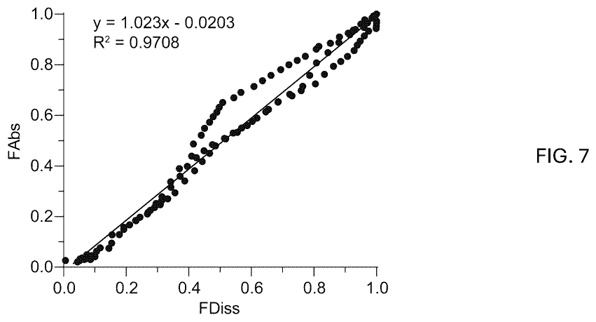

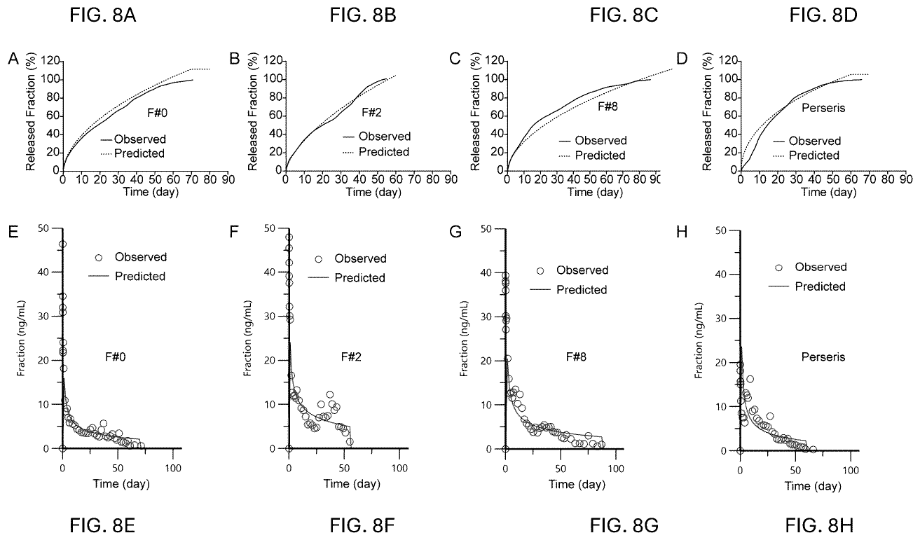

Formulations F #0, F #2, F #8 and the Reference Listed Drug (RLD), which showed desirable in vivo-in vitro consistency in drug release were used for IVIVC development. F #0, F #2 and F #8 were used for internal validation whereas the RLD was used for external validation. Makoid-Banakar and Weibull models were used to fit the dissolution profiles and the Fabs=Diss (Tvivo) model was utilized to build a correlation. Since the Makoid-Banakar and Weibull models gave very similar fitting for the dissolution profiles and the resultant IVIVCs were also similar, the data from the Makoid-Banakar model were presented as representative. The IVIVC linear regression between the in vitro percent of drug dissolution (X) and in vivo percent of drug absorption (Y) for these four formulations showed a fitting coefficient (R² value) of over 0.97 for both the Weibull and Makoid-Banakar models (see FIG. 7), indicating a high correlation. The IVIVC validation summaries using the Makoid-Banakar model for the in vitro release data are listed in Tables 3 and 4, respectively. The two IVIVC models showed very similar predictability. The internal predictability for both models was inconclusive as the average absolute percentage PE (% PE) for the Cₘₐₓ as well as the absolute % PE for Cₘₐₓ for each of the formulations were over the 10% acceptable limitation. The Cₘₐₓ was highly underestimated for all formulations. However, their external predictability was within 10% of the % PE for both the AUC and the Cₘₐₓ. This meets the FDA criteria for IVIVC establishment. As per the FDA guidance, the predictability of IVIVC is considered acceptable when the % PE for the Cₘₐₓ and AUC are lower than 15% for each formulation and the mean % PE values are lower than 10%. If these criteria are not met, external predictability of the IVIVC should be established for final determination (with % PE of 10% or less for both Cₘₐₓ and AUC). Accordingly, Level A IVIVCs were successfully developed using formulations F #0, F #2 and F #8 and their in vitro data obtained using the glass slide-based USP 2 method. The developed IVIVC was able to predict the entire in vivo performance from the in vitro data. This is confirmed by an overall good consistency between the predicted and observed in vivo profiles of all four formulations and between the predicted and observed PK profiles (see FIG. 8). This is the first time a level A IVIVC was successfully established for in situ forming implants.

Table 3 shows internal and external validation summary of IVIVC model of risperidone implants established using release data obtained from the glass slide adapter based USP 2 method. Fit dissolution model: Makoid-Banakar model; Correlation: Fabs-Diss (Tvivo); AUC unite: ng/mL*day; Cₘₐₓ unite: ng/mL.

TABLE 3

| Formulation | Parameter | Predicted | Observed | % PE |
|---|---|---|---|---|
| F#0 Internal | AUCₗₐₛₜ | 267.596 | 265.2278 | 0.892875 |
| F#0 Internal | Cₘₐₓ | 13.00765 | 46.41239 | −71.9737 |
| F#2 Internal | AUCₗₐₛₜ | 431.482 | 456.2263 | −5.42368 |
| F#2 Internal | Cₘₐₓ | 18.57361 | 47.97764 | −61.2869 |
| F#8 Internal | AUCₗₐₛₜ | 436.5053 | 453.3219 | −3.70965 |
| F#8 Internal | Cₘₐₓ | 17.60352 | 39.31681 | −55.2265 |
| Perseris External | AUCₗₐₛₜ | 282.5407 | 306.9998 | −7.96714 |
| Perseris External | Cₘₐₓ | 17.90538 | 19.54009 | −8.36595 |
| Avg Internal | AUCₗₐₛₜ | 378.5278 | 391.592 | 3.342068 |
| Avg Internal | Cₘₐₓ | 16.39493 | 44.56895 | 62.82906 |

Table 4 shows internal and external validation summary of IVIVC model of risperidone implants established using release data obtained from glass slide adapter based USP 2 method. Fit dissolution model: Weibull; Correlation: Fabs=Diss (Tvivo); AUC unite: ng/mL*day; Cₘₐₓ unite: ng/ml.

TABLE 4

| Formulation | Parameter | Predicted | Observed | % PE |
|---|---|---|---|---|
| F#0 Internal | AUCₗₐₛₜ | 269.0366 | 265.2278 | 1.436039 |
| F#0 Internal | Cₘₐₓ | 13.05009 | 46.41239 | −71.8823 |
| F#2 Internal | AUCₗₐₛₜ | 431.9479 | 456.2263 | −5.32156 |
| F#2 Internal | Cₘₐₓ | 18.2199 | 47.97764 | −62.0242 |
| F#8 Internal | AUCₗₐₛₜ | 438.1776 | 453.3219 | −3.34074 |
| F#8 Internal | Cₘₐₓ | 17.61458 | 39.31681 | −55.1984 |
| RLD External | AUCₗₐₛₜ | 283.7649 | 306.9998 | −7.56839 |
| RLD External | Cₘₐₓ | 18.36477 | 19.54009 | −6.01494 |
| Avg Internal | AUCₗₐₛₜ | 379.7207 | 391.592 | 3.366113 |
| Avg Internal | Cₘₐₓ | 16.29486 | 44.56895 | 63.03495 |

In an aspect, disclosed is a dissolution adapter including a slide/film with an open sandblasted receptacle for a sample placement, and a cassette. In an embodiment, the slide/film is a glass slide, a porcelain slide, a slide made from any other suitable material such as a polymer that can be given a rough surface, or a combination thereof. In an embodiment, the slide is a glass slide. In an embodiment, the slide is a porcelain slide. In an embodiment, the slide is made from a polymer such as regenerated cellulose, polytetrafluoroethylene, and the like.

In an embodiment, the surface of the slide including the open receptacle is sandblasted so that the formulation can stick well onto the slide during release testing. In an embodiment, only the open receptacle of the slide is sandblasted. In an embodiment, the open receptacle is a ring, a cylindrical well, or a round bottom concave. The open receptacle holds and controls the shape of a formulation that is tested. In an embodiment, the cassette is any suitable commercially available cassette, or any suitable customized cassette, for example, histology cassettes used for tissue embedding shown in FIG. 2.

In an embodiment, the slide is a disposable glass slide. In an embodiment, the dissolution adapter (herein also referred as “adapter”) is disposable. In an embodiment, the shape of the slide is adjustable. In an embodiment, the open receptacle has a diameter ranging from about 1.5 cm to about 8 cm. For example, the open receptacle has a diameter of about 0.8 cm, 1.6 cm, 1.8 cm, 2 cm, 2.2 cm, 2.4, cm, 2.6 cm, 2.8 cm, 3 cm, 3.2 cm, 3.4 cm, 3.6 cm, 3.8 cm, 4 cm, 4.2 cm, 4.4 cm, 4.6 cm, 4.8 cm, 5 cm, 5.2, cm, 5.4 cm, 5.6 cm, 5.8 cm, 6 cm, 6.2 cm, 6.4 cm, 6.6 cm, 6.8 cm, 7 cm, 7.2 cm, 7.4 cm, 7.6 cm, or about 7.8 cm. In an embodiment, the open receptacle has a depth ranging from about 0.1 mm to about 0.5 cm. For example, the open receptacle has the depth of about 0.2 mm, 0.3 mm, or 0.4 mm. In an embodiment, the open receptacle is a ring with a flat sandblasted surface. In an embodiment, the open receptacle is a sandblasted cylindrical well with depth of about 0.1 to 0.5 mm. In an embodiment, the open receptacle is a sandblasted round bottom concave with depth of about 0.1 to 0.5 mm.

In an embodiment, the dissolution adapter holds a sample such as in situ forming implant formulations in place and ensures reproducible shape of the formed implants. In an embodiment, the sample holding volume of the dissolution adapter is adjustable. In an embodiment, various surface-to-volume ratios of the samples can be achieved by the dissolution adapter through adjusting the size of the dissolution adapter receptacle or loading different volumes of the samples. In an embodiment, drug depots formed using this dissolution adapter are representative of the in vivo situation. In an embodiment, the formulations (for example, in situ forming implant formulations) tested in the dissolution adapter can be in the form of solutions, suspensions, or a combination thereof. In an embodiment, the formulations are in the form of solutions. In an embodiment, the formulations are in the form of suspensions. In an embodiment, the slide is resistant to organic solvents and/or chemicals which are commonly used in in situ forming implant formulations.

In certain embodiments, the dissolution adapters can be manufactured into different shapes and sizes to fit different needs and they can be used in different United States Pharmacopoeia (USP) dissolution apparati (for example, USP apparatus 2 and USP apparatus 4). In certain embodiments, the dissolution adapters are used in a screw capped bottles in a water bath shaker (non-Pharmacopocia dissolution apparatus) for the in vitro release testing of polymer-based in situ forming implants.

In an embodiment, the sample loading step with the disclosed dissolution adapters is simple and straightforward, saving time required for each sample loading into the adapters.

In an aspect, disclosed is a method for an in vitro release/dissolution testing of a sample (for example, a formulation) using the adapter disclosed herein, the method including: weighing the sample/formulation into the open receptacle of the slide/film such as the sandblasted glass slide; spreading the sample/formulation evenly; adding the slide into a cassette; immersing the adapter (the slide/film and cassette) into a dissolution medium of a testing apparati (such as a screw capped bottle in a water bath shaker, or a USP apparatus 4 flow-through cell); and detecting a drug concentration in the dissolution medium. In an embodiment, the slide is a glass slide, a porcelain slide, a slide/film made from any other suitable material such as a polymer that can be given a rough surface, or a combination thereof. In an embodiment, the slide is a glass slide. In an embodiment, the sample or formulation is an in situ forming implant formulation. In an embodiment, about 10 minutes of time is allowed for the sample to form a smooth surface before adding the slide into the cassette. In an embodiment, the sample (for example, in situ forming implant formulations) tested in the adapter can be in the form of solution or suspension.

The disclosed dissolution adapters herein offer the following advantages: the dissolution adapters are easy to fabricate and of low cost; the dissolution adapters improve the loading of formulations in place and ensures good reproducibility in terms of sample loading and its shape; the in-vitro dissolution study using this adapters show good reproducibility and discriminatory ability; the drug depots formed using these adapters are similar to that observed in the in vivo situation; and the release testing method may yield to successful Level A IVIVCs for tested formulations. The disclosed dissolution adapters can be also used in other USP dissolution apparatuses or non-compendial dissolution methods with minor adjustment of the dimensions. In general, the disclosed adapters ensure good reproducibility, discriminatory ability, and a biorelevant release profile of long-acting injectable in situ forming implant formulations.

The following terms are used to describe the invention of the present disclosure. In instances where a term is not specifically defined herein, that term is given an art-recognized meaning by those of ordinary skill applying that term in context to its use in describing the present disclosure.

A dissolution adapter is a specialized accessory used in pharmaceutical testing to hold a non-tablet dosage form (e.g., a film, gel, injectable depot, or implant) in place during in vitro dissolution testing. It ensures that the formulation maintains a defined surface area, orientation, and geometry when exposed to the dissolution medium, which makes the test more reproducible and comparable across studies. Disclosed herein is an improved in vitro release testing platform and a method for in situ forming implants that enable establishment of in vitro-in-vivo correlations (IVIVCs).

Perseris is a long-acting injectable formulation of risperidone, an atypical antipsychotic used to treat schizophrenia in adults.

Fabs=Diss (Tvivo) indicates the fraction dissolved as a function of an in vitro dissolution time (Tvivo).

In some embodiments, drug release or dissolution as a function of time t is modeled by a function, wherein the cumulative fraction released F (t) satisfies

$$F ( t ) = k {t}^{n} {e}^{- ct} ,$$

with k>0 a scale constant, n>0 an early-time shape exponent, and c≥0 a late-time attenuation constant; optionally, F(t) bounded by Fmax∈(0,1] such that 0≤F(t)≤Fmax. In certain embodiments, the MB parameters are determined by nonlinear regression to experimental in vitro data obtained from a formulation comprising a pharmaceutically acceptable therapeutic, a biodegradable polymer, and a solvent, under specified hydrodynamic and temperature conditions; goodness-of-fit may be confirmed by RMSE, AIC/BIC, and residual analysis. In some implementations, the MB model is applied over a validated time window [0, tᵥₐₗ] to capture fast onset with progressive damping, and may be used as the in-vitro input to an IVIVC framework after time-scaling and normalization to Fmax.

In some embodiments, drug release or dissolution is represented by a Weibull model having cumulative fraction released

$$F ( t ) = {F}_{max} ( 1 - exp [ - {( \frac{t − {T}_{i}}{α} )}^{β} ] ) \text{} ( t \geq {T}_{i} ) ,$$

where Fₘₐₓ∈(0,1] denotes the asymptotic fraction released, Ti≥0 is an optional lag time, α>0 is a scale parameter inversely related to release rate, and β>0 is a shape parameter such that β=1 yields first-order kinetics, β<1 yields concave-down early release, and β>1 yields sigmoidal behavior with an inflection point. In certain embodiments, (Fmax,Ti,α,β) are estimated by constrained nonlinear least squares with physically meaningful bounds 0≤F(t)≤Fmax; confidence intervals and lack-of-fit metrics are reported, and the model is used to (i) compare formulations, (ii) define dissolution specifications, and/or (iii) serve as the in vitro input for Level A IVIVC after appropriate time-mapping to in vivo absorption profiles.

In some embodiments, the apparent contact angle θapp of an in-situ forming pharmaceutical formulation comprising a pharmaceutically acceptable therapeutic molecule, a biodegradable polymer, and a solvent on a substrate having surface roughness characterized by an RMS amplitude Rq and correlation length ξ satisfies

$$cos {θ}_{app} \approx [ 1 + κ {( \frac{{R}_{q}}{ξ} )}^{2} ] \frac{{γ}_{SV} − {γ}_{SL}}{{γ}_{LV}} ,$$

where γ_{LV} is the liquid-vapor surface tension of the formulation, γ_{SV} and γ_{SL} are solid-vapor and solid-liquid interfacial tensions, respectively, and κ is a morphology constant determined for a family of surfaces prepared by the disclosed blasting or etching process.

A morphology constant (often written K is a unitless fitting parameter that captures how the shape and statistics of a surface's texture amplify the effect of roughness on wetting (or on area increase) beyond what's explained by a simple amplitude R_{q} and feature spacing ξ.

As used herein, the “morphology constant” (K) is a unitless, surface-family-specific parameter that characterizes how a substrate's topographical features-including asperity shape, RMS slope, anisotropy, and higher-order height statistics-amplify the effect of roughness on interfacial phenomena such as wetting and adhesion, beyond what is captured by simple amplitude and spacing descriptors. In certain embodiments, K relates the areal roughness factor

$$r = {A}_{true} / {A}_{proj}$$

to the root-mean-square roughness Rq and a lateral correlation length ξ according to a small-slope approximation:

$$r \approx 1 + κ {( {R}_{q} / ξ )}^{2}$$

accordingly, κ may be determined by profilometry or atomic force microscopy (AFM) mapping of the substrate to obtain Atrue, Aproj, Rq, and ξ followed by computation:

$$κ = ( r − 1 ) / {( {R}_{q} / ξ )}^{2}$$

using the same measurement bandwidth and filtering parameters. In some embodiments, K is used in a Wenzel-type wetting relation to predict the apparent contact angle of an in-situ forming pharmaceutical formulation comprising a biodegradable polymer, solvent, and therapeutic agent, and may be treated as constant for a class of surfaces prepared by a common process (e.g., bead-blasting, chemical etching, or laser texturing) while remaining independent of the liquid formulation. Representative ranges include κ≈0.5 to 3 for blasted, predominantly isotropic textures and κ≈2 to 10 for laser-textured, anisotropic microfeatures; however, K can be calibrated for a given surface family by fitting the foregoing relations to one or more reference measurements.

Unless otherwise defined, all technical and scientific terms used herein have the same meaning as commonly understood by one of ordinary skill in the art. For example, any nomenclature used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, are inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9% to 11% and “about 2%” means 1.8% to 2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human or a non-human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “effective amount” or “therapeutically effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose. As used herein, the term “prophylaxis” refers to preventing or reducing the progression of a disorder, either to a statistically significant degree or to a degree detectable by a person of ordinary skill in the art.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein. As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include 11C, 13C, and 14C. Accordingly, the compounds disclosed herein may include heavy or radioactive isotopes in the structure of the compounds or as substituents attached thereto. Examples of useful heavy or radioactive isotopes include 18F, 15N, 180, 76Br, 125I and 131I.

“Pharmaceutical compositions” means compositions comprising at least one active agent, such as a compound or salt of Formula (I), and at least one other substance, such as a carrier. Pharmaceutical compositions meet the U.S. FDA's GMP (good manufacturing practice) standards for human or non-human drugs.

A significant change is any detectable change that is statistically significant in a standard parametric test of statistical significance such as Student's T-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A film for receiving an implant formulation for in vitro testing, the film comprising:
a roughened surface;
a scribed area on the roughened surface for receiving an in situ forming implant formulation; the in situ formulation comprising a pharmaceutically acceptable therapeutic molecule, a biodegradable polymer, a water-miscible solvent, or any combination thereof; and
where a surface roughness of the roughened surface is operative to control a surface to volume ratio of the in situ forming implant formulation when tested in a dissolution adapter and/or a cassette such that a Level A in vitro-in vivo correlation is obtained.

2. The film of claim 1, wherein the film comprises a sandblasted glass slide, a porcelain slide, or a polymeric slide.

3. The film of claim 1, wherein the scribed area comprises an open receptacle, the open receptacle having a diameter of about 0.5 cm to about 8 cm.

4. The film of claim 3, wherein the open receptacle is selected from the group consisting of a created ring with a plane surface, a cylindrical well, or a round-bottom concave.

5. The film of claim 1, wherein the surface roughness has an average value Ra of about 0.1 to about 5 micrometers.

6. The film of claim 3, wherein the receptacle confines the in situ implant formulation to a defined geometry to control the surface-to-volume ratio exposed to the dissolution medium.

7. The film of claim 1, wherein the film comprises a ceramic, a polymer, or a combination thereof.

8. The film of claim 7, where the ceramic comprises a metal oxide, a metal carbide, a metal nitride, a metal boride, a metal silicide, a metal oxycarbide, a metal oxynitride, a metal boronitride, a metal carbonitride, a metal borocarbide, or a combination thereof.

9. The film of claim 8, where the metal oxide comprises silicon dioxide in the form of a glass slide.

10. The film of claim 7, where the polymer comprises a polyacrylic, a polycarbonate, a polyalkyd, a polystyrene, a polyolefin, a polyester, a polyamide, a polyaramid, a polyamideimide, a polyarylate, a polyurethane, an epoxy, a phenolic, a polysiloxane, a polyarylsulfone, a polyethersulfone, a polyphenylene sulfide, a polysulfone, a polyimide, a polyetherimide, a polytetrafluoroethylene, a polyetherketone, a polyether ether ketone, a polyether ketone ketone, a polybenzoxazole, a polyoxadiazole, a polybenzothiazole, a polybenzothiazinophenothiazine, a polypyrazinoquinoxaline, a polypyromellitimide, a polyguinoxaline, a polybenzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polycarborane, a polyoxabicyclononane, a polydibenzofuran, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polythioester, a polysulfone, a polysulfonamide, a polyurea, a polyphosphazene, a polysilazane, a polyolefin, or a combination thereof.

11. The film of claim 7, where the polymer comprises a polyolefin, a polytetrafluoroethylene, a polysiloxane, a polyester, or a combination thereof.

12. The film of claim 1, where the roughened surface is produced by abrasive blasting using sand, fine grit alumina, glass beads, or is produced by pore-forming technology, chemical etching, plasma etching, ion beam etching, or a combination thereof.

13. The film of claim 1, where the roughened surface controls a surface-to-volume ratio of the in situ forming implant formulation in a solidified form that is effective to closely mimic in vivo conditions.

14. The film of claim 1, where the roughened surface has a random texture or a periodic texture.

15. An article that contains the film of claim 1, where the article is a dissolution adapter or a cassette.

16. A method of establishing an in vitro-in vivo correlation (IVIVC) for a pharmaceutically acceptable therapeutic molecule, the method comprising:
disposing an in situ formulation comprising the pharmaceutically acceptable therapeutic molecule, a biodegradable polymer and a solvent into a roughened surface of a film, the roughened surface comprising a scribed receptacle that is operative to confine the formulation to a defined geometry;
disposing the film into a dissolution adapter where it is subjected to a dissolution solvent;
subjecting the dissolution adapter to in vitro release testing in a dissolution vessel selected from USP Apparatus 1, USP apparatus 2, USP Apparatus 4, or a screw-capped bottle in a water bath shaker, and collecting medium samples over time to determine the in vitro release profile;
administering the formulation to a subject and collecting plasma samples over time to determine an in vivo pharmacokinetic profile;
processing the in vivo pharmacokinetic profile by deconvolution to determine an in vivo absorption profile; and
correlating the in vitro release profile with the in vivo absorption profile to establish a Level A IVIVC.

17. The method of claim 16, wherein the dissolution medium comprises a buffered aqueous solution selected from phosphate-buffered saline, simulated body fluid, Hank's balanced salt solution, Tris buffer, acetate buffer, or carbonate buffer.

18. The method of claim 16, wherein the film comprises a sandblasted glass slide, a porcelain slide, or a polymeric slide.

19. The method of claim 16, where the roughened surface is produced by abrasive blasting using sand, fine grit alumina, glass beads, or is produced by pore-forming technology, chemical etching, plasma etching, ion beam etching, or a combination thereof.

20. The method of claim 16, where the roughened surface controls a surface-to-volume ratio of the in situ forming implant formulation in a solidified form that is effective to closely mimic in vivo conditions.
